(19) United States
(12) Patent Application Publication
(10) Pub. No.: US 2016/0073910 A1
(43) Pub. Date: Mar. 17, 2016

(54) System And Method For Thin Slice Acquisition Using Saturation Spin Labeling (TASSL) MR Angiography

(71) Applicant: Northshore University Healthsystem, Evanston, IL (US)

(72) Inventor: Robert R. Edelman, Highland Park, IL (US)

(21) Appl. No.: 14/852,678

(22) Filed: Sep. 14, 2015

Related U.S. Application Data
(60) Provisional application No. 62/049,659, filed on Sep. 12, 2014.

(51) Int. Cl. A61B 5/026; G01R 33/34; G01R 33/561; G01R 33/385; G01R 33/36

(52) U.S. Cl. CPC A61B 5/0263; G01R 33/385; G01R 33/3607; G01R 33/5611; G01R 33/34046; A61B 2576/00

(57) ABSTRACT

A system and method is provided for magnetic resonance angiography (MRA) that includes performing a labeling pulse to a labeling region having a first portion of a vascular system of a subject. The labeling pulse includes at least one excitation pulse and a slab-selective magnetic field gradient to saturate spins flowing from the labeling region and into an imaging region. The process also includes observing a delay period and performing an imaging pulse sequence to collect a label imaging data set from one or more views through the imaging region using an excitation pulse. The preceding is repeated with a TR selected to ensure that the spins flowing within the imaging region are kept substantially saturated during a majority of repetitions. The process also includes acquiring a non-labeling imaging data set without saturating spins and reconstructing an image using the labeling imaging data set and the non-labeling imaging data.

### CROSS-REFERENCE TO RELATED APPLICATIONS

This application is based on, claims priority to, and incorporates herein by reference in its entirety, U.S. Provisional Application Ser. No. 62/049,659, filed Sep. 12, 2014, and entitled “System And Method For Thin Slice Acquisition Using Saturation Spin Labeling (TASSL) MR Angiography.”

### BACKGROUND OF THE INVENTION

When a substance such as human tissue is subjected to a uniform magnetic field (polarizing field B₀), the individual magnetic moments of the nuclear spins in the tissue attempt to align with this polarizing field, but precess about it in random order at their characteristic Larmor frequency. Usually the nuclear spins are comprised of hydrogen atoms, but other NMR active nuclei are occasionally used. A net magnetic moment M_{z} is produced in the direction of the polarizing field, but the randomly oriented magnetic components in the perpendicular, or transverse, plane (x-y plane) cancel one another. If, however, the substance, or tissue, is subjected to a magnetic field (excitation field B₁; also referred to as the radiofrequency (RF) field) which is in the x-y plane and which is near the Larmor frequency, the net aligned moment, M_{z}, may be rotated, or “tipped” into the x-y plane to produce a net transverse magnetic moment Mₜ, which is rotating, or spinning, in the x-y plane at the Larmor frequency. The practical value of this phenomenon resides in the signal which is emitted by the excited spins after the excitation field B₁ is terminated. There are a wide variety of measurement sequences in which this nuclear magnetic resonance (NMR) phenomenon is exploited.

When utilizing these signals to produce images, magnetic field gradients (Gₓ, G_{y}, and G_{z}) are employed. Typically, the region to be imaged experiences a sequence of measurement cycles in which these gradients vary according to the particular localization method being used. The emitted MR signals are detected using a receiver coil. The MRI signals are then digitized and processed to reconstruct the image using one of many well-known reconstruction techniques.

Magnetic resonance angiography (MRA) and, related imaging techniques, such as perfusion imaging, use the NMR phenomenon to produce images of the human vasculature or physiological performance related to the human vasculature. There are three main categories of techniques for achieving the desired contrast for the purpose of MR angiography. The first general category is typically referred to as contrast enhanced (CE) MRA. The second general category is phase contrast (PC) MRA. The third general category is time-of-flight (TOF) or tagging-based MRA.

To perform CE MRA, a contrast agent, such as gadolinium, is injected into the patient prior to the magnetic resonance (MR) angiogram to enhance the diagnostic capability of the MR angiogram. Perfusion imaging is employed to assess the viability of tissues. A contrast agent is administered to the subject and a series of MR images are acquired as the contrast agent perfuses into the tissues of interest. From this series of contrast-enhanced MR images hemodynamic parameters such as blood flow, blood volume, and mean transit time may be computed.

While CE MRA is a highly effective means for noninvasively evaluating the vascular and physiological performance, for example, by studying perfusion, the technique suffers from several additional drawbacks. First, the contrast agent that must be administered to enhance the blood vessel carries a significant financial cost. Second, contrast agents such as gadolinium have recently been shown to be causative of a debilitating and potentially fatal disorder called nephrogenic systemic fibrosis (NSF). Third, CE MRA, may not provide accurate or sufficient hemodynamic information, so that it is not always feasible to determine if a stenosis is hemodynamically significant or to asses the perfusion in a clinically useful manner.

As such, non-contrast enhanced (NE) MRA methods have become more prevalent. Phase contrast (PC) MRA is largely reserved for the measurement of flow velocities and imaging of veins. Phase contrast sequences are the basis of MRA techniques utilizing the change in the phase shifts of the flowing protons in the region of interest to create an image. Spins that are moving along the direction of a magnetic field gradient receive a phase shift proportional to their velocity. Specifically, in a PC MRA pulse sequence, two data sets with a different amounts of flow sensitivity are acquired. This is usually accomplished by applying gradient pairs, which sequentially dephase and then rephase spins during the sequence. The first data set is acquired using a “flow-compensated” pulse sequence or a pulse sequence without sensitivity to flow. The second data set is acquired using a pulse sequence designed to be sensitive to flow. The amount of flow sensitivity is controlled by the strength of the bipolar gradient pairs used in the pulse sequence because stationary tissue undergoes no effective phase change after the application of the two gradients, whereas the different spatial localization of flowing blood is subjected to the variation of the bipolar gradient. Accordingly, moving spins experience a phase shift. The raw data from the two data sets are subtracted to yield images that illustrate the phase change, which is proportional to spatial velocity.

To perform PC MRA pulse sequences, a substantial scan time is generally required and the operator must set a velocity-encoding sensitivity, which varies unpredictably depending on a variety of clinical factors. Additionally, PC imaging requires pre-selection of velocity encoding sensitivity and specialized processing of the phase-information of the MR images. The latter is prone to errors stemming from phase aliasing, random phase in regions of low signal intensity, and eddy current effects. It is not reliable for depicting veins with very slow or absent flow.

Fortunately, TOF imaging techniques do not require the use of a contrast agent and do not rely on potentially-precarious velocity encoding sensitivities. Contrary to CE-MRA, which relies on the administered contrast agent to provide an increase in measured MR signal, TOF MRA relies on the inflow of blood into an imaging volume to increase the signal intensity of the vasculature as compared to the stationary background tissues. This is achieved by the application of a number of RF excitation pulses to the imaging volume that cause the magnetization of the stationary background tissues to reach a saturation value. Since inflowing blood entering the imaging volume is not exposed to the same number of RF excitation, it will provide higher MR signal intensity than the background tissue. The differences between the signal intensity of the stationary background tissues and the inflowing blood thus provide a contrast mechanism exploited by TOF MRA.

Three dimensional (3D) TOF methods work well for the carotid bifurcation and intracranial circulation, but do not work well in the peripheral arterial circulation because flow velocities are insufficient to adequate refresh saturated spins within the thick 3D imaging slab. Two dimensional (2D) TOF methods require cardiac gating when applied to the peripheral arterial circulation or suffer from pulsation artifacts. Moreover, with 2D TOF, a large flip angle is required to produce sufficient contrast between inflowing blood in arteries and background tissues.

In an effort to increase contrast attributable to the relatively small signal levels or weight particular signals, for example, those attributable to cerebral blood flow (CBF) or another measurable mechanism, various “tagging” or “labeling” methods have been developed. One such method is referred to as the arterial spin labeling (ASL) family of techniques.

Although mostly used for perfusion imaging, there are a few reports of using ASL for other applications. Previously-described ASL methods for MR angiography generally rely on a 3D acquisition. While 3D ASL methods have the benefit of providing excellent suppression of signal from stationary background tissues, they cannot be used to image the peripheral arterial circulation because flow is triphasic and velocities are relatively low. Thus, the labeled or tagged spins move too slowly and/or move back as well as forward through the arteries and confound results of the ASL techniques. That is, 3D ASL methods invert the arterial spins and use a long inflow delay, which is incompatible with imaging of the peripheral vascular structures. The long inflow delay also greatly increases the time between pulse sequence repetitions and thereby prolongs the scan time.

Often, nonenhanced MRA methods as well as labeling- or tagging-based angiography methods must be synchronized with the cardiac cycle to avoid artifacts, particularly for imaging of the peripheral arteries. To do so, electrocardiogram (ECG) leads are attached to the patient and the acquired ECG waveform is used to gate the imaging process. Applying ECG leads increases setup time for the patient. Moreover, in patients with a highly irregular heart rhythm, image quality may be compromised due to the gating not being well synchronized with the irregular hart rhythm.

Therefore, it would be desirable to have a system and method for performing angiographic studies using MRI systems without the drawbacks presented by CE-MRA or traditional NE-MRA methods.

### SUMMARY OF THE INVENTION

The present invention provides systems and methods for producing an angiogram with a magnetic resonance imaging (MRI) system without requiring the use of exogenous contrast agents, the placement of ECG leads, scan parameters tailored to an individual patient's physiology, or extended scan times.

In accordance with one aspect of the present disclosure, a system and method is provided that includes acquiring a magnetic resonance angiography (MRA) image by performing a labeling pulse to a labeling region having a first portion of a vascular system of a subject extending through the labeling region to label spins moving within the first labeling region. The labeling pulse includes at least one excitation pulse in combination with a slab-selective magnetic field gradient to saturate spins flowing from the labeling region and into an imaging region extending, as a non-limiting example, less than 32 mm along a direction of flow of the spins flowing from the labeling region into the imaging region. The process also includes observing a delay period that is less than, for example, a T1 relaxation time of the spins flowing from the labeling region into the imaging region. The process also includes performing an imaging pulse sequence to collecting a label imaging data set from one or more views through the imaging region using an excitation pulse with a value of a flip angle that is, for example, less than twice a value of a repetition time (TR) of the imaging pulse sequence. The preceding may be repeated with a TR selected to allow the spins flowing within the imaging region to be kept substantially saturated during a majority of repetitions. The processes may also include acquiring a non-labeling imaging data set from the imaging region without saturating spins flowing from the labeling region and into the imaging region and reconstructing an image using the labeling imaging data set and the non-labeling imaging data set to create a magnetic resonance angiography image.

In accordance with another aspect of the present disclosure, a magnetic resonance imaging (MRI) system is provided that includes a magnet system configured to generate a polarizing magnetic field about at least a portion of a subject arranged in the MRI system, a plurality of gradient coils configured to apply a gradient field to the polarizing magnetic field, and a radio frequency (RF) system configured to apply an excitation field to the subject and acquire MR image data therefrom. The MRI system also includes a computer system programmed to control the plurality of gradient coils and the RF system according to a labeling pulse sequence to a labeling region having a first portion of a vascular system of a subject extending through the labeling region to label spins moving within the first labeling region, wherein the labeling pulse sequence includes at least one excitation pulse in combination with a slab-selective magnetic field gradient to saturate spins flowing from the labeling region and into an imaging region extending, for example, less than 32 mm along a direction of flow of the spins flowing from the labeling region into the imaging region. The computer system is also programmed to observe a delay period that may be less than a T1 relaxation time of the spins flowing from the labeling region into the imaging region and control the plurality of gradient coils and the RF system according an imaging pulse sequence to collecting an imaging data set from one or more views through the imaging region using an excitation pulse with a value of a flip angle that may be, for example, less than twice a value of a repetition time (TR) of the imaging pulse sequence. The computer system is further programmed to control the plurality of gradient coils and the RF system to repeat at least the preceding steps with a TR selected to ensure that the spins flowing within the imaging region are kept substantially saturated during a majority of repetitions. The computer system is further programmed to control the plurality of gradient coils and the RF system to acquire a non-labeling imaging data set from the imaging region without saturating spins flowing from the labeling region and into the imaging region and reconstruct an image using the labeling imaging data set and the non-labeling imaging data set to create a magnetic resonance angiography image.

The foregoing and other advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of an MRI system for use with the present invention.

FIG. 2 is a schematic representation of a transceiver system for use with the MRI system of FIG. 1.

FIG. 3 is a flow chart of the steps performed in accordance with one exemplary implementation of the present invention.

### DETAILED DESCRIPTION OF THE INVENTION

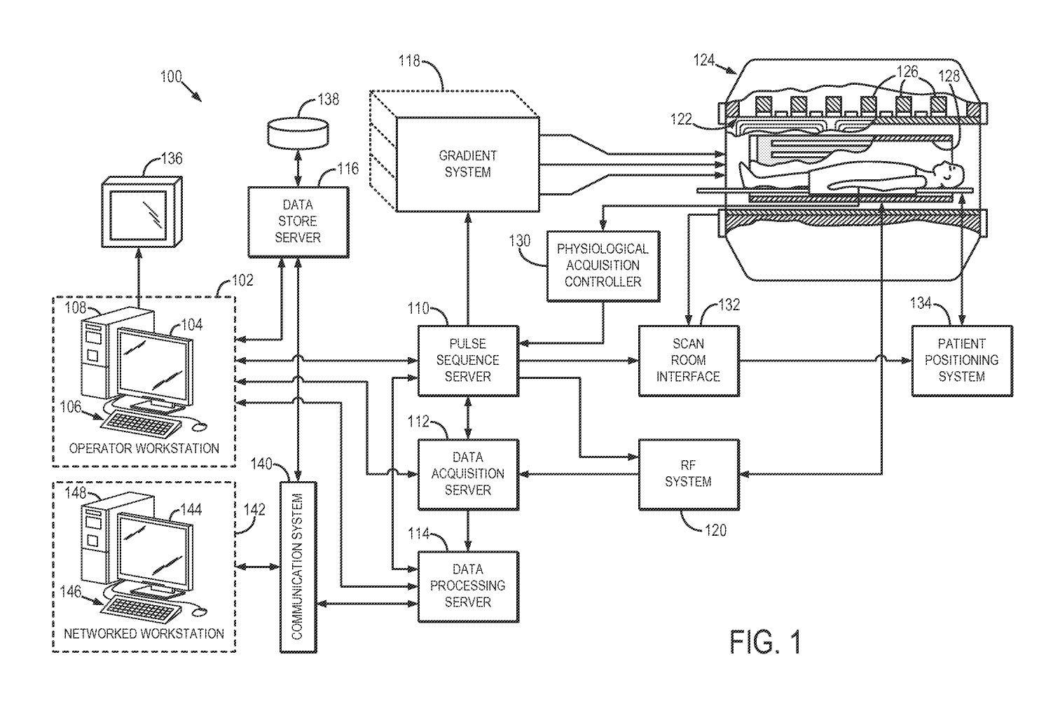

Referring particularly to FIG. 1, an example of a magnetic resonance imaging (MRI) system 100 is illustrated. The MRI system 100 includes a workstation 102 having a display 104 and a keyboard 106. The workstation 102 includes a processor 108 that is commercially available to run a commercially-available operating system. The workstation 102 provides the operator interface that enables scan prescriptions to be entered into the MRI system 100. The workstation 102 is coupled to four servers: a pulse sequence server 110; a data acquisition server 112; a data processing server 114; and a data store server 116. The workstation 102 and each server 110, 112, 114, and 116 are connected to communicate with each other.

The pulse sequence server 110 functions in response to instructions downloaded from the workstation 102 to operate a gradient system 118 and a radiofrequency (RF) system 120. Gradient waveforms necessary to perform the prescribed scan are produced and applied to the gradient system 118, which excites gradient coils in an assembly 122 to produce the magnetic field gradients Gₓ, G_{y}, and G_{z} used for position encoding MR signals. The gradient coil assembly 122 forms part of a magnet assembly 124 that includes a polarizing magnet 126 and a whole-body RF coil 128 (or a head (and neck) RF coil for brain imaging).

RF excitation waveforms are applied to the RF coil 128, or a separate local coil, such as a head coil, by the RF system 120 to perform the prescribed magnetic resonance pulse sequence. Responsive MR signals detected by the RF coil 128, or a separate local coil, are received by the RF system 120, amplified, demodulated, filtered, and digitized under direction of commands produced by the pulse sequence server 110. The RF system 120 includes an RF transmitter for producing a wide variety of RF pulses used in MR pulse sequences. The RF transmitter is responsive to the scan prescription and direction from the pulse sequence server 110 to produce RF pulses of the desired frequency, phase, and pulse amplitude waveform. The generated RF pulses may be applied to the whole body RF coil 128 or to one or more local coils or coil arrays.

The RF system 120 also includes one or more RF receiver channels. Each RF receiver channel includes an RF preamplifier that amplifies the MR signal received by the coil 128 to which it is connected, and a detector that detects and digitizes the quadrature components of the received MR signal. The magnitude of the received MR signal may thus be determined at any sampled point by the square root of the sum of the squares of the I and Q components:

M=\sqrt{I²+Q²} (1)

and the phase of the received MR signal may also be determined:

$$ϕ = {tan}^{- 1} ( \frac{Q}{I} ) . \qquad ( 2 )$$

The pulse sequence server 110 also optionally receives patient data from a physiological acquisition controller 130. The controller 130 receives signals from a number of different sensors connected to the patient, such as electrocardiograph (ECG) signals from electrodes, or respiratory signals from a bellows or other respiratory monitoring device. Such signals are typically used by the pulse sequence server 110 to synchronize, or “gate,” the performance of the scan with the subject's heart beat or respiration.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized MR signal samples produced by the RF system 120 are received by the data acquisition server 112. The data acquisition server 112 operates in response to instructions downloaded from the workstation 102 to receive the real-time MR data and provide buffer storage, such that no data is lost by data overrun. In some scans, the data acquisition server 112 does little more than pass the acquired MR data to the data processor server 114. However, in scans that require information derived from acquired MR data to control the further performance of the scan, the data acquisition server 112 is programmed to produce such information and convey it to the pulse sequence server 110. For example, during prescans, MR data is acquired and used to calibrate the pulse sequence performed by the pulse sequence server 110. Also, navigator signals may be acquired during a scan and used to adjust the operating parameters of the RF system 120 or the gradient system 118, or to control the view order in which k-space is sampled. In all these examples, the data acquisition server 112 acquires MR data and processes it in real-time to produce information that is used to control the scan.

The data processing server 114 receives MR data from the data acquisition server 112 and processes it in accordance with instructions downloaded from the workstation 102. Such processing may include, for example: Fourier transformation of raw k-space MR data to produce two or three-dimensional images; the application of filters to a reconstructed image; the performance of a backprojection image reconstruction of acquired MR data; the generation of functional MR images; and the calculation of motion or flow images.

Images reconstructed by the data processing server 114 are conveyed back to the workstation 102 where they are stored. Real-time images are stored in a data base memory cache (not shown), from which they may be output to operator display 112 or a display 136 that is located near the magnet assembly 124 for use by attending physicians. Batch mode images or selected real time images are stored in a host database on disc storage 138. When such images have been reconstructed and transferred to storage, the data processing server 114 notifies the data store server 116 on the workstation 102. The workstation 102 may be used by an operator to archive the images, produce films, or send the images via a network or communication system 140 to other facilities that may include other networked workstations 142.

The communications system 140 and networked workstation 142 may represent any of the variety of local and remote computer systems that may be included within a given clinical or research facility including the system 100 or other, remote location that can communicate with the system 100. In this regard, the networked workstation 142 may be functionally and capably similar or equivalent to the operator workstation 102, despite being located remotely and communicating over the communication system 140. As such, the networked workstation 142 may have a display 144 and a keyboard 146. The networked workstation 142 includes a processor 148 that is commercially available to run a commercially-available operating system. The networked workstation 142 may be able to provide the operator interface that enables scan prescriptions to be entered into the MRI system 100.

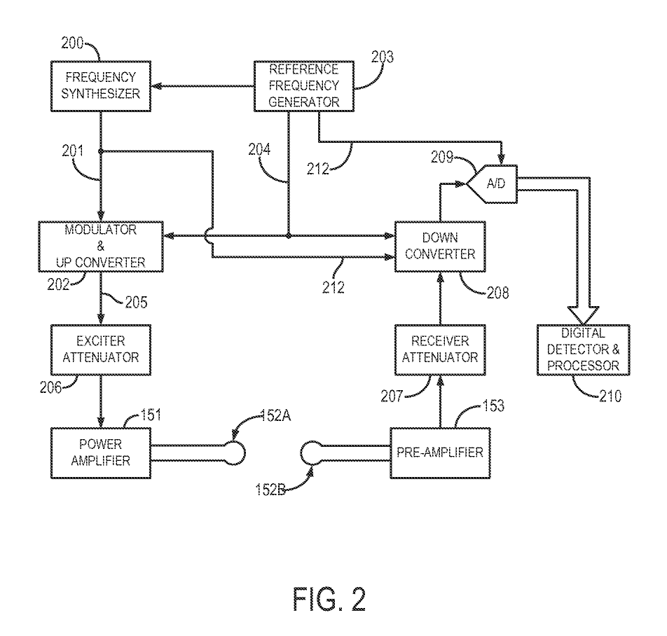

As shown in FIG. 1, the RF system 26 may be connected to the whole body RF coil 34, or as shown in FIG. 2, a transmitter section of the RF system 26 may connect to one RF coil 151A and its receiver section may connect to a separate RF receive coil 151B. Often, the transmitter section is connected to the whole body RF coil 34 and each receiver section is connected to a separate local coil 151B.

Referring particularly to FIG. 2, the RF system 26 includes a transmitter that produces a prescribed RF excitation field. The base, or carrier, frequency of this RF excitation field is produced under control of a frequency synthesizer 200 that receives a set of digital signals from the pulse sequence server 18. These digital signals indicate the frequency and phase of the RF carrier signal produced at an output 201. The RF carrier is applied to a modulator and up converter 202 where its amplitude is modulated in response to a signal R(t) also received from the pulse sequence server 18. The signal R(t) defines the envelope of the RF excitation pulse to be produced and is produced by sequentially reading out a series of stored digital values. These stored digital values may, be changed to enable any desired RF pulse envelope to be produced.

The magnitude of the RF excitation pulse produced at output 205 is attenuated by an exciter attenuator circuit 206 that receives a digital command from the pulse sequence server 18. The attenuated RF excitation pulses are applied to the power amplifier 151 that drives the RF coil 151A.

Referring still to FIG. 2, the signal produced by the subject is received by the receiver coil 152B and applied through a preamplifier 153 to the input of a receiver attenuator 207. The receiver attenuator 207 further amplifies the signal by an amount determined by a digital attenuation signal received from the pulse sequence server 18. The received signal is at or around the Larmor frequency, and this high frequency signal is down converted in a two step process by a down converter 208 that first mixes the NMR signal with the carrier signal on line 201 and then mixes the resulting difference signal with a reference signal on line 204. The down converted NMR signal is applied to the input of an analog-to-digital (ND) converter 209 that samples and digitizes the analog signal and applies it to a digital detector and signal processor 210 to produce the I values and Q values corresponding to the received signal. As described above, the resulting stream of digitized I and Q values of the received signal are output to the data acquisition server 20 of FIG. 1. The reference signal, as well as the sampling signal applied to the ND converter 209, is produced by a reference frequency generator 203.

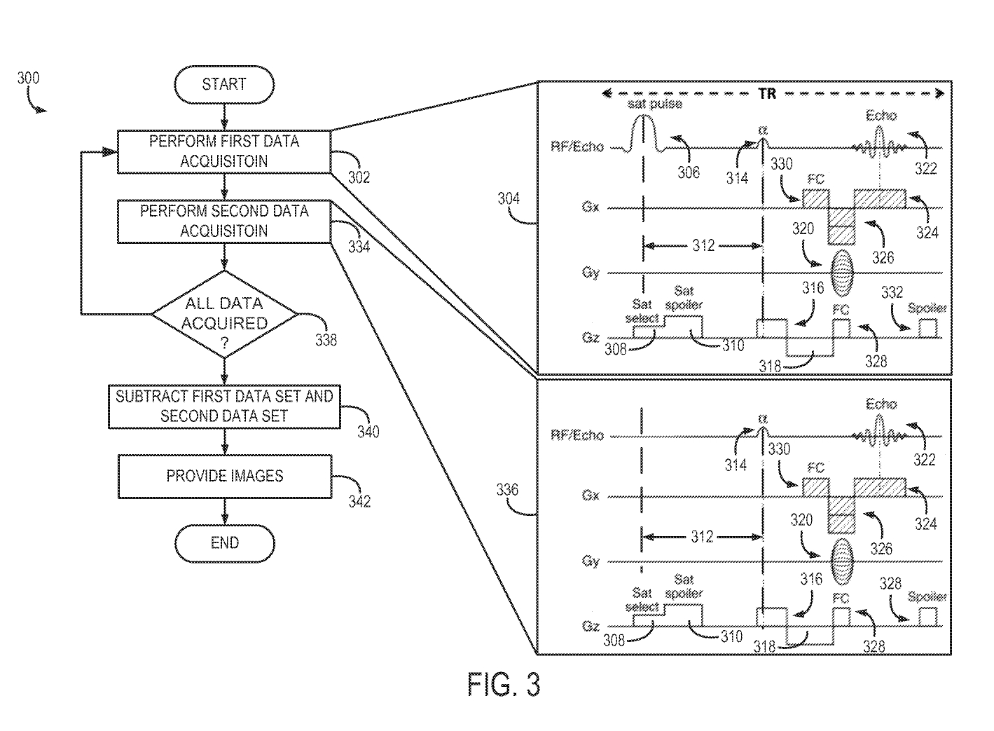

Referring to FIG. 3, a method 300 in accordance with the present disclosure a first data acquisition is performed at process block 302. As illustrated in the associated pulse sequence diagram 304, a pulse sequence is performed that, as will be described, includes is repeated over a plurality of repetition times (TR) to acquire a stack of thin two-dimensional (2D) slices. That is, as will be further described, the pulse sequence may be a spoiled gradient-echo pulse sequence with a low flip angle excitation, which targets signal from the static blood while controlling significant saturation effects.

The pulse sequence 304 includes a saturation RF pulse 306 that is performed in coordination with saturation selection and saturation spoiler gradients 308, 310. As will be further explained, the saturation pulse 306 is applied frequently, and at least faster than a T1 relaxation time of blood. As a non-limiting example, the saturation pulse 306 may be applied once every 100 ms, and a time delay 312 between the application of the saturation pulse 306 and application of a subsequent excitation of an imaging slice with an excitation pulse 314 is controlled to be very small. As a non-limiting example, the time delay may be less than 100 ms. Furthermore, the excitation pulse 314 may have a small flip angle to, as will be explained, control against saturation of downstream arterial spins. For example, the flip angle may be selected to be less than twice a TR of the pulse sequence 304. As a non-limiting example, the flip angle may be approximately 20 degrees or less for a TR of 18 ms.

The RF excitation pulse 314 is played out in the presence of a slice-selective gradient 316 in order to produce transverse magnetization in a prescribed imaging slice. The slice selective gradient 316 includes a rephasing lobe 318 that acts to rephase unwanted phase accruals caused by the RF excitation pulse 316. Following excitation of the nuclear spins in the prescribed imaging slice, a phase encoding gradient 320 is applied to spatially encode a nuclear magnetic resonance signal, representative of a gradient-recalled echo 322, along one direction in the prescribed imaging slice. A readout gradient 324 is also applied after a dephasing gradient lobe 326 to spatially encode the signal representative of the echo 322 along a second, orthogonal direction in the prescribed imaging slice. In addition, flow compensation (FC) gradients are applied 328, 330 along with the above-described gradients. Finally, a spoiler gradient 332 is played out along the phase-select gradient axis in order to prepare magnetization for subsequent repetitions of the pulse sequence.

The above-described imaging process uses particular constraints for imaging parameters to make clinically useful images. That is, the present disclosure recognizes that, unlike traditional imaging methods, a balance of a series of competing constraints can be achieved to yield images that, otherwise, would be marred with artifacts that would destroy the clinical utility of the images.

In particular, for the image set acquired using the first pulse sequence 304 including an RF saturation pulse 306, the saturation pulse 306 is repeated sufficiently often, and the thickness of the imaging slice is sufficiently thin, that spins within the slice are constantly in a saturated state. As a non-limiting example, each set of acquired data includes a stack of thin, for example, 1-4 mm, two-dimensional (2D) slices. Consequently, the saturation pulse 306 is applied frequently. As a non-limiting example, the saturation pulse 306 may be applied at least once every 100 ms.

Also, the time delay 312 between the application of the saturation pulse 306 and excitation of imaging slice with the excitation pulse 314 may be controlled to small. As one non-limiting example, the time delay 312 may be less than 100 ms. Furthermore, the imaging slice should be desirably thin. In one non-limiting example, the imaging slice may be less than 32 mm and, as another non-limiting example, may be less than 10 mm.

If any of these constraints are not balanced, then unsaturated spins can flow into the imaging slice or in-slice spins can recover their longitudinal magnetization, which the present disclosure recognizes will cause signal loss after subtraction of the two image sets. This is in distinction to traditional arterial spin labeling and tagging methods for MR angiography, which use a thick 3D imaging slab, instead of multiple thin slices, an inversion RF pulse or pseudocontinuous train of RF pulses producing spin inversion, instead of spin saturation, a long TR that may be 2-3 seconds long, and a long time delay between the application of each inversion pulse and the acquisition of the imaging volume, such as 1 second or longer.

Both sets of images are may be acquired using, for example, the above described variation on a spoiled gradient-echo pulse sequence. However, the excitation pulse 314 may have a low flip angle, for example, less than twice the TR of the sequence or around 20 degrees for a duration of 20 or less ms, and is used to ensure a high signal from the static blood, while controlling any significant saturation effects. This stands in contrast with traditional 2D time of flight methods that use a large flip angle because the present disclosure recognizes that in such traditional methods the arterial spins downstream from the imaging slice become saturated and, during periods of flow reversal, which occurs due to the triphasic flow pattern in peripheral arteries, these saturated spins will flow back into the imaging slice and cause artifacts.

Referring again to the flow chart illustrating the method 300, once the first acquisition is performed, a second acquisition is performed at process block 334. In this case, as illustrated, generally, at 336, the above-described pulse sequence is repeated, however, without the saturation pulse 306. That is, for the first acquisition at process block 302 a low-compensated (FC) 2D spoiled gradient pulse sequence 304 is performed that includes an arterial saturation pulse applied once every TR. For the second acquisition at process block 330, the pulse sequence 336 performed is identical to the prior pulse sequence 306, except that no saturation RF pulse 306 is applied.

At decision block 338, the process reiterates until all data has been acquired. There after, the data set formed from repetition of the first data acquisition at process block 302 and the data set formed from repetition of the second data acquisition at process block 334 is subtracted at process block 340. In the images created from the first data acquisition at process block 302, arteries appear dark while veins and background tissue appear bright. In the images created from the second data acquisition at process block 334 where no saturation pulses are applied, arteries, veins, and background tissue appear bright. Subtraction of the two image sets at process block 340 produces images in which arteries appear bright, while veins and background tissue appear dark. At process block 342, the final angiogram images may be provided. For example, a maximum intensity projection algorithm may be used to create an angiogram.

The present invention has been described in terms of one or more preferred embodiments, and it should be appreciated that many equivalents, alternatives, variations, and modifications, aside from those expressly stated, are possible and within the scope of the invention.

1. A method for acquiring a magnetic resonance angiography (MRA) image of a portion of a vascular system of a subject using a magnetic resonance imaging system, the method including steps comprising:
i) performing a labeling pulse to a labeling region having a first portion of a vascular system of a subject extending through the labeling region to label spins moving within the first labeling region, wherein the labeling pulse includes at least one excitation pulse in combination with a slab-selective magnetic field gradient to saturate spins flowing from the labeling region and into an imaging region extending less than 32 mm along a direction of flow of the spins flowing from the labeling region into the imaging region;
ii) observing a delay period that is less than a T1 relaxation time of the spins flowing from the labeling region into the imaging region;
iii) performing an imaging pulse sequence to collecting a label imaging data set from one or more views through the imaging region using an excitation pulse with a value of a flip angle that is less than twice a value of a repetition time (TR) of the imaging pulse sequence; and
iv) repeating at least steps i) through iii) with a TR selected to ensure that the spins flowing within the imaging region are kept substantially saturated during a majority of repetitions of step iii);
v) acquiring a non-labeling imaging data set from the imaging region without saturating spins flowing from the labeling region and into the imaging region;
vi) reconstructing an image using the labeling imaging data set and the non-labeling imaging data set to create a magnetic resonance angiography image.

2. The method of claim 1 wherein the delay period is less than 100 ms.

3. The method of claim 1 wherein step iv) is performed to image a stack of at least one of two-dimensional (2D) slices or three-dimensional (3D) slices.

4. The method of claim 1 wherein the imaging slice is less than 10 mm along the direction of flow of the spins flowing from the labeling region into the imaging region.

5. The method of claim 1 wherein the imaging slice is less than 1 mm along the direction of flow of the spins flowing from the labeling region into the imaging region to control against unsaturated spins flowing into the imaging region.

6. The method of claim 1 wherein the excitation pulse has flip angle of less than 25 degrees.

7. The method of claim 1 wherein the excitation pulse is played out in less than 20 ms.

8. The method of claim 1 wherein step v) includes applying a spatially non-selective RF pulse to balance magnetization transfer effects from the labeling pulse applied before step v).

9. The method of claim 1 wherein step v) includes performing an RF pulse applied to saturate venous spins in the imaging slice.

10. The method of claim 1 wherein step v) is performed immediately after each acquisition of the label imaging data set and before step iv).

11. The method of claim 1 where step v) is performed before steps i) through iii).

12. The method of claim 1 further comprising undersampling k-space or performing an accelerated imaging technique to acquire at least one of the label imaging data set and non-label imaging data set according to at least one of GRAPPA imaging, SENSE imaging, partial Fourier imaging, reduced field of view imaging, or radial undersampling imaging.

13. The method of claim 1 wherein the flip angle is less than 90 degrees.

14. The method of claim 1 wherein the flip angle is greater than 90 degrees but less than 180 degrees.

15. The method of claim 1 wherein step vi) includes subtracting the labeling imaging data set and the non-labeling imaging data set to create a subtraction data set and performing a projection of the subtraction data set to create the magnetic resonance angiography image.

16. The method of claim 1 wherein arteries within the magnetic resonance angiography image appear bright and other tissues appear dark.

17. A magnetic resonance imaging (MRI) system comprising:
a magnet system configured to generate a polarizing magnetic field about at least a portion of a subject arranged in the MRI system;
a plurality of gradient coils configured to apply a gradient field to the polarizing magnetic field;
a radio frequency (RF) system configured to apply an excitation field to the subject and acquire MR image data therefrom;
a computer system programmed to:
i) control the plurality of gradient coils and the RF system according to a labeling pulse sequence to a labeling region having a first portion of a vascular system of a subject extending through the labeling region to label spins moving within the first labeling region, wherein the labeling pulse sequence includes at least one excitation pulse in combination with a slab-selective magnetic field gradient to saturate spins flowing from the labeling region and into an imaging region extending less than 32 mm along a direction of flow of the spins flowing from the labeling region into the imaging region;
ii) observe a delay period that is less than a T1 relaxation time of the spins flowing from the labeling region into the imaging region;
iii) control the plurality of gradient coils and the RF system according an imaging pulse sequence to collecting an imaging data set from one or more views through the imaging region using an excitation pulse with a value of a flip angle that is less than twice a value of a repetition time (TR) of the imaging pulse sequence;
iv) control the plurality of gradient coils and the RF system to repeat at least steps i) through iii) with a TR selected to ensure that the spins flowing within the imaging region are kept substantially saturated during a majority of repetitions of step iii);
v) control the plurality of gradient coils and the RF system to acquire a non-labeling imaging data set from the imaging region without saturating spins flowing from the labeling region and into the imaging region; and
vi) reconstruct an image using the labeling imaging data set and the non-labeling imaging data set to create a magnetic resonance angiography image.

18. The system of claim 17 the delay period is less than 100 ms.

19. The system of claim 17 wherein step iv) is performed to image a stack of 2D slices.

20. The system of claim 17 wherein the imaging slice is less than 10 mm along the direction of flow of the spins flowing from the labeling region into the imaging region.

21. The system of claim 17 wherein the imaging slice is less than 1 mm along the direction of flow of the spins flowing from the labeling region into the imaging region to control against unsaturated spins flowing into the imaging region.

22. The system of claim 17 wherein the excitation pulse has flip angle of less than 25 degrees.

23. The system of claim 17 wherein the excitation pulse is played out in less than 20 ms.
